(19) United States
(12) Patent Application Publication
(10) Pub. No.: US 2025/0391020 A1
(43) Pub. Date: Dec. 25, 2025

(54) MOTION ROBUST CARDIOVASCULAR IMAGING

(71) Applicant: Ohio State Innovation Foundation, Columbus, OH (US)

(72) Inventors: Rizwan Ahmad, Columbus, OH (US); Syed Murtaza Arshad, Columbus, OH (US)

(21) Appl. No.: 19/249,490

(22) Filed: Jun. 25, 2025

Related U.S. Application Data
(60) Provisional application No. 63/663,874, filed on Jun. 25, 2024.

(51) Int. Cl. G06T 7/00; A61B 5/00; A61B 5/055; A61B 5/08; G06T 7/20

(52) U.S. Cl. CPC G06T 7/0012; A61B 5/0044; A61B 5/055; A61B 5/0816; G06T 7/20; G06T 2207/10088

(57) ABSTRACT

An example computer-implemented method for image reconstruction, includes: receiving k-space data from a magnetic resonance imaging (MRI) machine, the k-space data comprising a plurality of k-space readouts; sorting the k-space readouts into a set of bins comprising binned k-space data, each of the set of bins corresponding to a respective phase of a respiratory cycle; iteratively performing the steps of: (i) computing a soft participation weight for each k-space readout, where the soft participation weight reflects a posterior probability that each k-space readout belongs to each of the set of bins; and (ii) updating an image estimate by solving a weighted optimization problem; determining a convergence criterion is reached; and outputting a motion-resolved volumetric MRI image when the convergence criterion is reached.

### CROSS-REFERENCE TO RELATED APPLICATIONS

This application claims the benefit of U.S. provisional patent application No. 63/663,874, filed on Jun. 25, 2024, and titled “MOTION ROBUST CARDIOVASCULAR IMAGING,” the disclosure of which is expressly incorporated herein by reference in its entirety.

### STATEMENT REGARDING FEDERALLY FUNDED RESEARCH

This invention was made with government support under R01 HL151697 awarded by the National Institutes of Health. The government has certain rights in the invention.

### BACKGROUND

Cardiovascular imaging can be used in a variety of diagnosis and treatment contexts. One type of cardiovascular magnetic resonance imaging (CMR) is volumetric CMR that collects data under free-breathing conditions. CMR collects data in k-space and uses the k-space data to reconstruct a CMR image. Improvements to CMR can improve systems and methods of medical imaging.

### SUMMARY

In some aspects, implementations of the present disclosure include a computer-implemented method for image reconstruction, the method including: receiving k-space data from a magnetic resonance imaging (MRI) machine, the k-space data including a plurality of k-space readouts; sorting the k-space readouts into a plurality of bins including binned k-space data, each of the plurality of bins corresponding to a respective phase of a cardiac and respiratory cycle; iteratively performing the steps of: (i) computing a soft participation weight for each k-space readout, wherein the soft participation weight reflects a posterior probability that each k-space readout belongs to each of the plurality of bins; and (ii) updating an image estimate by solving a weighted optimization problem; determining when a convergence criterion is reached; and outputting a motion-resolved volumetric MRI image when the convergence criterion is reached. 2.

In some aspects, implementations of the present disclosure include a computer-implemented method, wherein step (ii) includes fixed ADMM iterations.

In some aspects, implementations of the present disclosure include a computer-implemented method, wherein the convergence criterion includes a maximum number of ADMM iterations.

In some aspects, implementations of the present disclosure include a computer-implemented method, wherein the convergence criterion includes a threshold of normalized squared image difference between iterations of steps (i) and (ii). 6. 7. 8. 9.

In some aspects, implementations of the present disclosure include a computer-implemented method, wherein the k-space data includes motion artifacts originating from respiratory, cardiac, or bulk motion.

In some aspects, implementations of the present disclosure include a computer-implemented method, wherein the plurality of k-space readouts are acquired by self-gating readouts.

In some aspects, implementations of the present disclosure include a system including: an MRI machine; a controller operably coupled to the MRI machine, wherein the controller includes a processor and a memory, wherein the memory has non-transitory computer-readable instructions stored thereon, that, when executed by the processor, cause the processor to: receive k-space data from a magnetic resonance imaging (MRI) machine, the k-space data including a plurality of k-space readouts; sort the k-space readouts into a plurality of bins including binned k-space data, each of the plurality of bins corresponding to a respective phase of a respiratory cycle; iteratively performing the steps of: (i) computing a soft participation weight for each k-space readout, wherein the soft participation weight reflects a posterior probability that each k-space readout belongs to each of the plurality of bins; and (ii) updating an image estimate by solving a weighted optimization problem; determining a convergence criterion is reached; and output a motion-resolved volumetric MRI image when the convergence criterion is reached.

In some aspects, implementations of the present disclosure include a system, wherein step (ii) includes fixed ADMM iterations.

In some aspects, implementations of the present disclosure include a system, wherein the convergence criterion includes a maximum number of ADMM iterations.

In some aspects, implementations of the present disclosure include a system, wherein the convergence criterion includes a threshold of normalized squared image difference between iterations of steps (i) and (ii).

In some aspects, implementations of the present disclosure include a system, wherein the k-space data includes motion artifacts originating from respiratory, cardiac, or bulk motion.

In some aspects, implementations of the present disclosure include a system, wherein the plurality of k-space readouts are acquired by self-gating readouts.

In some aspects, implementations of the present disclosure include a system, wherein the system further includes a graphical user interface configured to display the motion-resolved volumetric MRI image.

In some aspects, implementations of the present disclosure include a system, wherein the system further includes a remote computing device, and wherein the instructions further cause the processor to transmit the motion-resolved volumetric MRI image to the remote computing device.

In some aspects, implementations of the present disclosure include a non-transitory computer-readable medium having instructions thereon, that, when executed by a processor, cause the processor to: receive k-space data from a magnetic resonance imaging (MRI) machine, the k-space data including a plurality of k-space readouts; sort the k-space readouts into a plurality of bins including binned k-space data, each of the plurality of bins corresponding to a respective phase of a respiratory cycle; iteratively performing the steps of: (i) computing a soft participation weight for each k-space readout, wherein the soft participation weight reflects a posterior probability that each k-space readout belongs to each of the plurality of bins; and (ii) updating an image estimate by solving a weighted optimization problem; determine a convergence criterion is reached; and output a motion-resolved volumetric MRI image when a convergence criterion is reached.

In some aspects, implementations of the present disclosure include a non-transitory computer-readable medium, wherein step (ii) includes 12 ADMM iterations.

In some aspects, implementations of the present disclosure include a non-transitory computer-readable medium, wherein the convergence criterion includes a maximum number of ADMM iterations.

In some aspects, implementations of the present disclosure include a non-transitory computer-readable medium, wherein the convergence criterion includes a threshold of normalized squared image difference between iterations of steps (i) and (ii).

In some aspects, implementations of the present disclosure include a non-transitory computer-readable medium, wherein the k-space data includes motion artifacts originating from respiratory, cardiac, or bulk motion.

In some aspects, implementations of the present disclosure include a non-transitory computer-readable medium, wherein the plurality of k-space readouts are acquired by self-gating readouts.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

FIG. 1A illustrates an example method of performing image reconstruction, according to implementations of the present disclosure.

FIG. 1B illustrates an example system for performing image reconstruction, according to implementations of the present disclosure.

FIG. 2A illustrates a method of image reconstruction, according to implementations of the present disclosure.

FIG. 2B illustrates a method of preprocessing data that can be used with the method of 2A.

FIG. 3 illustrates an example computing device.

FIGS. 4A-4D illustrate a quantitative comparison showing improvements of an example implementation of the present disclosure compared to conventional compressed sensing (CS), where FIG. 4A illustrates a Peak Signal-to-Noise Ratio (PSNR) comparison, FIG. 4B illustrates an Structural Similarity Index (SSIM) comparison, FIG. 4C illustrates an edge sharpness comparison, and FIG. 4D illustrates a Brier Score comparison.

FIG. 5 illustrates example short-axis slices obtained by an example implementation of the present disclosure, compared with short-axis slices obtained by a conventional CS method, showing the improvements of the example implementation of the present disclosure.

FIG. 6 illustrates example respiratory surrogate signals overlayed with simulated bulk motion intervals, according to a study of an example implementation of the present disclosure.

FIG. 7 illustrates representative sagittal cine frames reconstructed using an example implementation of the present disclosure compared to a conventional CS method, showing the improvements of the example implementation of the present disclosure.

FIG. 8 illustrates respiratory surrogate signals overlaid with the assignment percentage to an outlier bin, according to a study of an example implementation of the present disclosure.

The components in the drawings are not necessarily to scale relative to each other. Like reference numerals designate corresponding parts throughout the several views.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for Cardiovascular MRI, it will become evident to those skilled in the art that the implementations are not limited thereto, but are applicable for various MRI and imaging applications.

MRI systems and methods are commonly used in the diagnosis and treatment of many different conditions. MRI systems and methods often acquire images over a period of time, which can cause motion artifacts based on movement of the region of interest relative to the MRI system during the acquisition of the images. The k-space data acquired is used to reconstruct a 2-D or 3-D medical image. Motion artifacts can degrade the performance of MRI systems and methods, including the resulting 2-D or 3-D images produced. Implementations of the present disclosure include improved systems and methods for reconstructing images from k-space data, for example k-space data including motion artifacts.

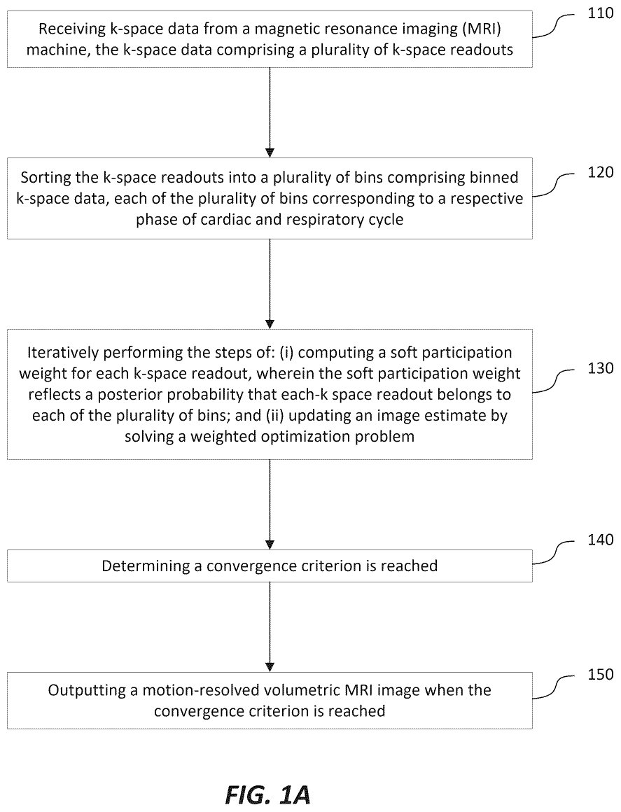

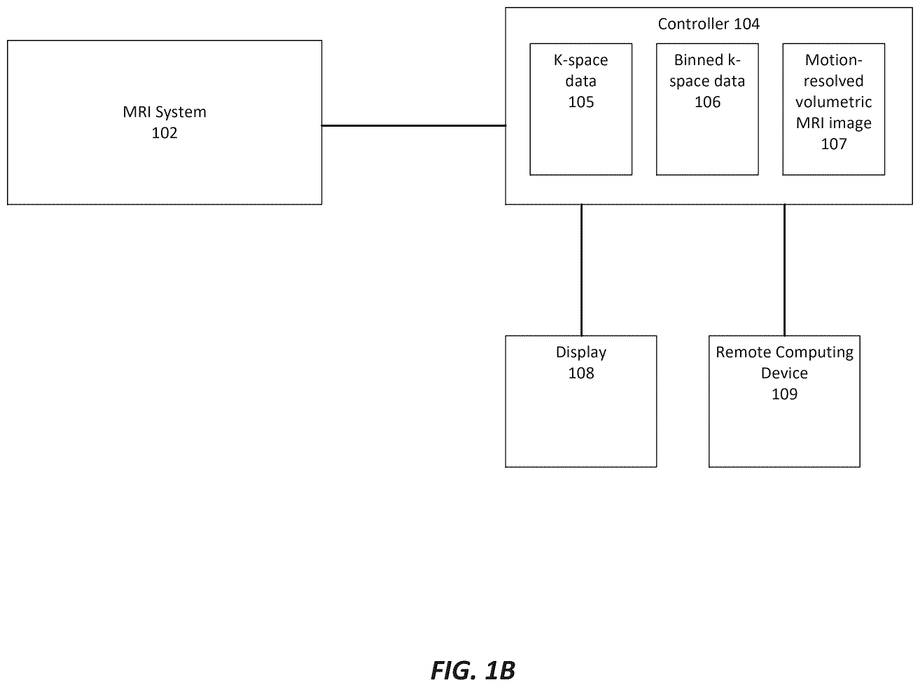

With reference to FIG. 1A, an example method for image reconstruction is shown according to implementations of the present disclosure.

At step 110, the method includes receiving k-space data from a magnetic resonance imaging (MRI) machine. The k-space data can include a set of k-space readouts. Optionally, the k-space readouts are acquired with self-gating readouts. The k-space data can include k-space data that is captured sequentially in time (e.g., cine data), and therefore the k-space data can include artifacts originating from respiratory, cardiac, bulk motion, and/or any other type of motion.

At step 120, the method includes sorting the k-space readouts into a plurality of bins comprising binned k-space data, where each of the bins correspond to a respective phase of a cardiac and respiratory cycle.

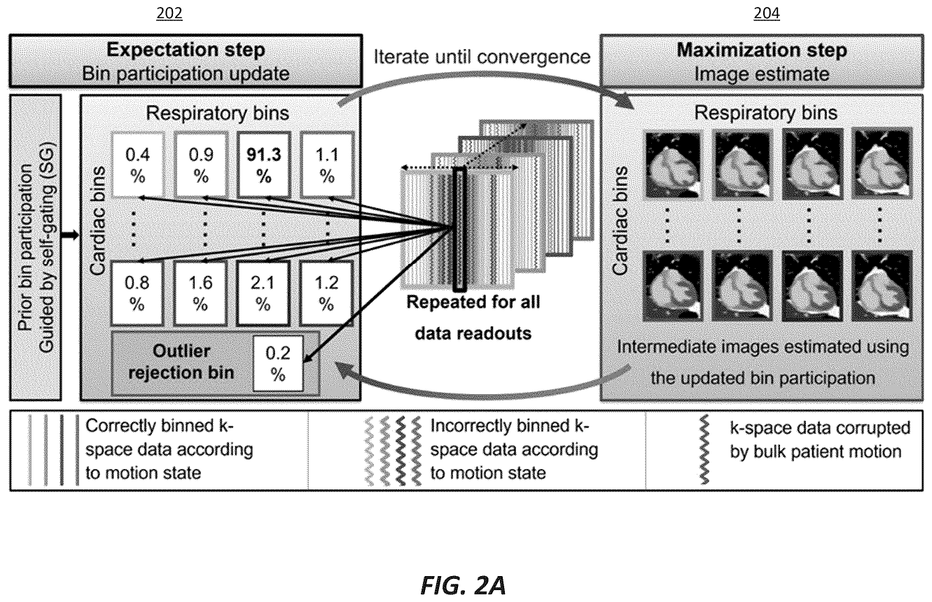

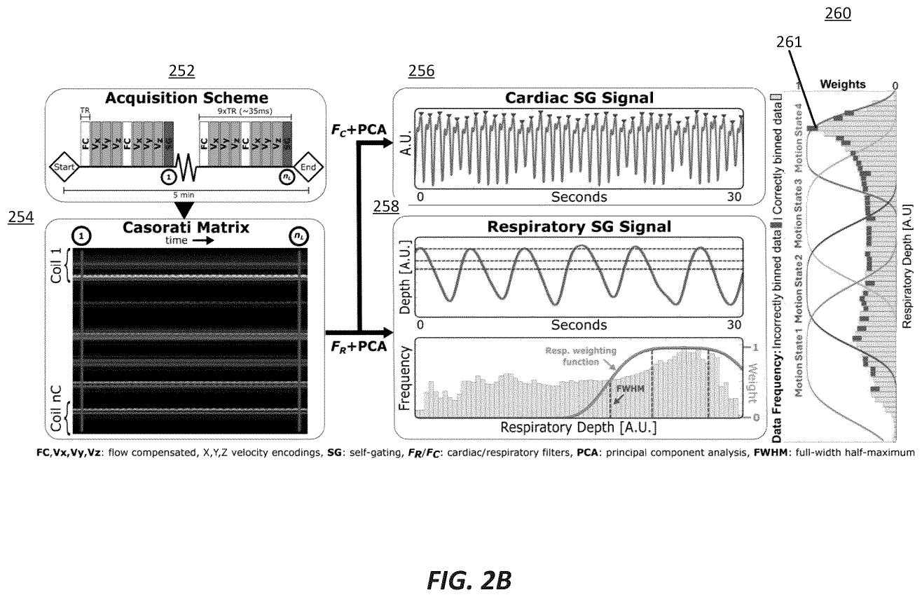

At step 130, the method includes iteratively performing the steps of: (i) computing a soft participation weight for each k-space readout, wherein the soft participation weight reflects a posterior probability that each k-space readout belongs to each of the plurality of bins; and (ii) updating an image estimate by solving a weighted optimization problem. As described further with reference to the “Example” herein, step i can be referred to as the “E-step” and step ii can be referred to as the “M-step.” Further description of the E-step 202 and M-step 204 is provided with reference to FIG. 2A herein. Optionally, the steps i and ii of step 130 can be repeated any number of times.

Optionally, step (ii) can include performing fixed ADMM iterations. Additional description of fixed ADMM iterations is provided in the example hereto.

At step 140, the method can include determining a convergence criterion is reached. Once the convergence criterion is reached, the motion-resolved volumetric MRI image can be suitable for output.

At step 150, the method includes outputting a motion-resolved volumetric MRI image when the convergence criterion is reached. The convergence criterion can optionally be based on the number of ADMM iterations. Alternatively or additionally, the convergence criterion can be based on a threshold of normalized squared image difference between iterations of steps (i) and (ii). In yet additional example implementations, multiple convergence criteria can be used, where the iteration of steps i and ii is configured to stop when the first convergence criterion of the multiple convergence criteria is met.

FIG. 1B illustrates an example block diagram of a system that can be used to implement the methods described herein. The system can include an MRI system 102, a controller 104, a display 108, and a remote computing device 109. The MRI system 102 can optionally be in wired or wireless communication with the controller 104 to transmit k-space data 105 (e.g., any number of k-space readouts) to the controller 104. The controller 104 can further be configured to store the binned k-space data 106 and the motion-resolved volumetric MRI image 107 created according to the method of FIG. 1A.

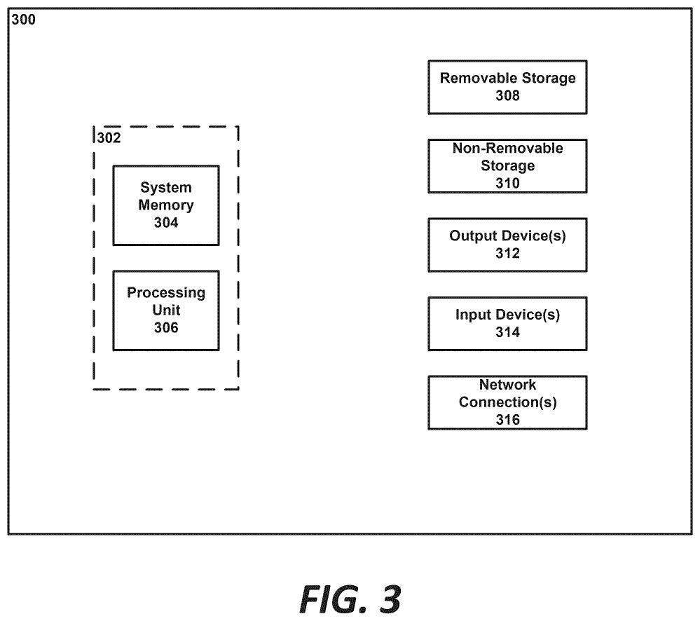

The controller 104 can include any or all of the features of the example computing device 300 shown in FIG. 3. The controller 104 can optionally output the motion-resolved volumetric MRI image 107 to the display 108 for viewing, and/or to a remote computing device 109.

FIG. 2A illustrates an example method of image reconstruction according to implementations of the present disclosure, and FIG. 2B illustrates a method of preprocessing data that can be used with the method of 2A. As shown in FIG. 2A, the method includes an E-step 202 to refine bin participation of readouts to valid motion bins and an outlier bin, given the prior bin participation and current image estimate. In the M-step 204, the image estimate is improved using the refined bin participation. Both steps are repeated until convergence, resulting in motion-compensated images.

The example implementation includes an expectation maximization (EM)-based approach where the initial bin assignment for each k-space readout is iteratively refined during the reconstruction processing, which results in motion artifact reduction. The example implementation can optionally be configured so that data that do not belong to any of the valid cardiac/respiratory motion states is assigned to a “outlier” bin. This can benefit cases where some of the data are corrupted due to bulk motion. Although EM algorithms have been applied to different applications, this is the first extension of EM to volumetric CMR.

In some implementations of the present disclosure, the steps of estimating the state of the image and updating the binned k-space data can be iteratively performed. For example, the steps can be iteratively performed based on a threshold or other measure of the accuracy of the reconstructed image. As a non-limiting example, maximizing posterior probability of the image can be used to determine the reconstructed image based on the updated bin assignment of the k-space data.

Alternatively or additionally, the present disclosure contemplates that outlier rejection can be performed (e.g., rejecting outliers of the k-space data). In some implementations, performing outlier rejection can include assigning k-space data to an outlier bin.

As shown in FIG. 2B, an example self-gating signal extraction and processing pipeline can include acquisition at step 252. At step 254, the acquired self-gating readout lines are reorganized into Casorati matrix C. Two parallel filtering operations 256, 258 are performed along the temporal dimension (rows) of C, followed by PCA to extract cardiac and respiratory motion surrogate signals. Simulated soft distribution of data can be output at step 260, with sections of bars 261 representing residual respiratory motion. In FIG. 2B, AU represents arbitrary units.

As used herein, the terms “about” or “approximately” when referring to a measurable value such as an amount, a percentage, and the like, are meant to encompass variations of ±20%, ±10%, ±5%, or ±1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

It should be appreciated that the logical operations described herein with respect to the various figures may be implemented (1) as a sequence of computer implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 3, an example computing device 300 upon which the methods described herein may be implemented is illustrated. It should be understood that the example computing device 300 is only one example of a suitable computing environment upon which the methods described herein may be implemented. Optionally, the computing device 300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random access memory (RAM)), non-volatile (such as read-only memory (ROM), flash memory, etc.), or some combination of the two. This most basic configuration is illustrated in FIG. 3 by box 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media and non-removable media implemented in any method or technology for storage of information such as computer readable instructions, data structures, program modules or other data. System memory 304, removable storage 308, and non-removable storage 310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

A study was performed of an example implementation of the present disclosure entitled EMORe. EMORe includes an adaptive reconstruction method configured to enhance motion robustness in free-running, free-breathing self-gated 5D cardiac magnetic resonance imaging. Traditional self-gating-based motion binning for 5D MRI often results in residual motion artifacts due to inaccuracies in cardiac and respiratory signal extraction and sporadic bulk motion, compromising clinical utility. EMORe addresses these issues by integrating adaptive inter-bin correction and explicit outlier rejection within an expectation maximization (EM) framework, where the E-step and M-step are executed alternately until convergence. In the E-step, probabilistic (soft) bin assignments are refined by correcting misassignment of valid data and rejecting motion-corrupted data to a dedicated outlier bin. In the M-step, the image estimate is improved using the refined soft bin assignments. Validation in a simulated 5D MRXCAT phantom demonstrated EMORe's superior performance compared to standard compressed sensing reconstruction, showing significant improvements in peak signal-to-noise ratio, structural similarity index, edge sharpness, and bin assignment accuracy across varying levels of simulated bulk motion. The study included in-vivo validation in 13 volunteers, which confirmed EMORe's robustness, significantly enhancing blood-myocardium edge sharpness and reducing motion artifacts compared to compressed sensing, particularly in scenarios with controlled coughing-induced motion. EMORe's adaptability and robust handling of bulk motion artifacts significantly enhances the clinical applicability and diagnostic confidence of 5D cardiac MRI. The robustness derives from adaptive, probabilistic, retrospective data binning and outlier rejection implemented in an expectation-maximization framework. The EMORe approach adaptively mitigates binning inaccuracies caused either by motion estimation techniques or by sporadic bulk patient motion. For 3D imaging resolved in both cardiac and respiratory phases, the technique offers improved image sharpness and motion artifact suppression, compared to conventional methods.

### Example Implementation

EM-Guided Binning Correction and Outlier Rejection (EMORe).

In FRV-CMR, the acquired N data readouts are retrospectively assigned to K target cardiorespiratory motion bins using self-gating (SG). However, as discussed herein the initial SG-based bin assignments of N acquired readouts to K motion states, or bins, is inherently imperfect. Let gₙ ∈ {1, . . . , K} denote the SGbased bin assignment for the n^{th} readout. To mitigate inaccurate data binning, the proposed EMORe framework leverages the EM algorithm by treating the true motion bin assignments as latent random variables, represented by rₙ ∈{1, . . . , K, K+1}, where the additional (K+1)^{th} bin corresponds to an outlier bin for discarding readouts corrupted by bulk motion that do not belong to any valid motion bin {1, . . . , K}. The algorithm learns the probabilities that a readout belongs to any bin, given the k-space data and the current image estimates; a readout then participates in image formation for any motion state bin weighted by these probabilities.

The EMORe algorithm iteratively performs two steps until convergence: the E-step 202 and the M-step 204 as shown in FIG. 2A. In the E-step 202, EMORe refines the probabilistic (soft) participation of acquired readouts across the valid motion bins and outlier bin based on the current estimates of the images for all motion states. Subsequently, the M step 204updates the image estimates utilizing the refined bin participation obtained from the E-step. By integrating explicit inter-bin correction and outlier rejection within this EM framework, EMORe enhances robustness against incorrect binning, bulk motion and other types of data corruption, thereby significantly improving the quality of reconstructed images. Detailed implementations of the Estep and M-step in the EMORe framework are herein.

E-step: In the E-step of the EMORe framework, the current estimate of the images and the acquired data are used to update the probabilistic (soft) participation weights for each readout across all K+1 motion bins. The participation weight of the n^{th} readout to k^{th} bin at iteration t, denoted

$${w}_{( n , k )}^{( t )} ,$$

represents the posterior provability of the readout belonging to the bin. Via Bayes' theorem these weights are given by:

$$\begin{matrix}{w}_{( n , k )}^{( t )} = p ( {r}_{n} = k \vert {y}_{n} , {\hat{x}}^{( t - 1 )} ) \\ = \frac{p ( {y}_{n} \vert {\hat{x}}^{( t - 1 )} , {r}_{n} = k ) \cdot p ( {r}_{n} = k \vert {\hat{x}}^{( t - 1 )} )}{{\sum }_{j = 1}^{K + 1} p ( {y}_{n} \vert {\hat{x}}^{( t - 1 )} , {r}_{n} = j ) \cdot p ( {r}_{n} = j \vert {\hat{x}}^{( t - 1 )} )}\end{matrix}$$

Assuming that the prior probability of the n^{th} readout belonging to k^{th} bin, p (rₙ=k)=θ_{(n,k)} is independent of the image estimate,

$${w}_{( n , k )}^{( t )} = \frac{p ( {y}_{n} \vert {\hat{x}}^{( t - 1 )} , {r}_{n} = k ) \cdot {θ}_{( n , k )}}{{\sum }_{j = 1}^{K + 1} p ( {y}_{n} \vert {\hat{x}}^{( t - 1 )} , {r}_{n} = j ) \cdot {θ}_{( n , j )}} \qquad ( 1 a )$$

For valid motion bins (k=1, . . . , K),

$$p ( {y}_{n} \vert {\hat{x}}^{( t - 1 )} , {r}_{n} = k ) = \frac{exp ( - \frac{1}{L {σ}^{2}} {‖ {A}_{( n , k )} {\hat{x}}^{( t - 1 )} - {y}_{n} ‖}_{2}^{2} )}{{πσ}^{2}} \qquad ( 1 b )$$

and, for the outlier motion bin (k=K+1),

$$p ( {y}_{n} \vert {\hat{x}}^{( t - 1 )} , {r}_{n} = K + 1 ) = \frac{1}{π {σ}^{2}} exp ( - \frac{{τ}^{2}}{{σ}^{2}} ) . \qquad ( 1 c )$$

Here, A_{(n,k)} ∈ [symbol] ^{L×M} is the forward operator that incorporates pixel-wise multiplication with coil sensitivity maps, Fourier transform, and selection of the n^{th} readout assigned to the k^{th} motion state bin. Additionally, σ is the standard deviation of the circularly symmetric Gaussian noise in the measured data, L is the length of each coil-sensitive readout vector, and M is the number of voxels in the volumetric image for each motion state. Given that the duration of a single readout is negligible relative to physiological motion, the study assumed motion affects the entire readout vector yₙ and normalize the squared residual norm by Lin (1b) to estimate probabilities per readout. The parameter τ determines the degree of outlier data rejection: a smaller value of τ corresponds to more aggressive outlier rejection at the potential cost of discarding valid data. The choice of bin prior probabilities, θ_{(n,k)}, is also described herein.

M-step: In the M-step of the EMORe framework, the updated readout participation weights

$${w}_{( n , k )}^{( t )}$$

are used to re-estimate the images \hat{x}^{(t)}. For additive white Gaussian measurement noise, this step minimizes the negative logposterior probability of the images x given the readout measurements yₙ, updated weights

$${w}_{( m . k )}^{( t )} ,$$

and a regularizing prior proportional to [symbol] The corresponding optimization problem is formulated as:

$${\hat{x}}^{( t )} = arg {min}_{x} {\sum }_{n = 1}^{N} {\sum }_{k = 1}^{K} [ - {w}_{( n , k )}^{( t )} log p ( {y}_{n} \vert {r}_{n} = k , x ) ] + ℛ ( x ) = arg {min}_{x} {\sum }_{n = 1}^{N} {\sum }_{k = 1}^{K} [ \frac{{w}_{( n , k )}^{( t )}}{L {σ}^{2}} {‖ {A}_{( n , k )} x - {y}_{n} ‖}_{2}^{2} ] + {λ}_{s} {‖ {∇}_{s} x ‖}_{1} + {λ}_{c} {‖ {∇}_{c} x ‖}_{1} + {λ}_{r} {‖ {∇}_{r} x ‖}_{1} . \qquad ( 2 )$$

where V̅ₛ, V̅_{c}, and V̅ᵣ denote the anisotropic total variation (TV) operator enforcing smoothness along three spatial dimensions, the cardiac temporal dimension, and the respiratory temporal dimension, respectively. The hyperparameters λₛ, λ_{c}, and λᵣ control the strengths of regularization in their respective dimensions. The optimization problem in (2) is solved using the alternating direction method of multipliers (ADMM) [36]. In (2), the data fidelity term is normalized by L to maintain consistency with (1b).

Algorithm parameters: The complete EMORe algorithm for FRV-CMR reconstruction is presented in Algorithm 1. The EM algorithm is sensitive to initialization [34], so the example implementation initializes \hat{x}⁽⁰⁾ by partially solving (2) for I₁ ADMM iterations, using initial readout participation weights derived from SG-based bin assignment as follows,

$${w}_{( n , k )}^{( 0 )} = \{ \begin{matrix}1 , & if k = {g}_{n} , \\ 0 , & otherwise\end{matrix} . ∀ n , k \qquad ( 3 )$$

While this SG-based assignment is imperfect, it provides adequate initialization for convergence of the algorithm to a reasonable local minimum.

In the high-dimensional inverse problem of FRV-CMR, the EM algorithm can become unstable [37], especially when the total number data bins K is large (e.g., K=80, for 20 cardiac×4 respiratory bins). To mitigate this instability, the example implementation uses a bin-assignment prior θ_{(n,k)}, where

$${\sum }_{k = 1}^{K + 1} {θ}_{( n , k )} = 1$$

for every n. An informative prior guided by SG-based initial assignments is defined as follows:

$${θ}_{( n , k )} = \{ \begin{matrix}{α}_{g} , & if k = {g}_{n} \\ {α}_{0} , & if k = K + 1 \\ ( 1 - {α}_{g} - {α}_{o} ) / ( K - 1 ) , & otherwise\end{matrix} \qquad ( 4 )$$

The hyperparameters α_{g} and αₒ are probabilities, chosen to balance the effectiveness of residual motion compensation against algorithm convergence stability. Specifically, a higher value of α_{g} increases reliance on the initial SGbased binning, enhancing stability but limiting motion correction. Whereas, a higher value of αₒ promotes aggressive rejection of outliers, at the potential cost of discarding valid data.

The M-step presents a large-scale optimization problem due to the large size of each volumetric image. Therefore, obtaining a closed-form solution or fully solving the M-step problem through iterative procedures is computationally expensive and impractical. To overcome this, the example implementation uses a generalized EM (GEM) approach, where the M-step is approximately solved via I₂ ADMM iterations. The stopping criterion of the EMORe algorithm is either the maximum allowed number of ADMM iterations, denoted by J, or a threshold η on the normalized squared image difference between consecutive EM iterations whichever is achieved first. Example algorithm hyperparameters are given in Table I. These values were optimized to achieve the best results on one digital subject of the phantom study.

### Algorithm 1 EMORe Reconstruction

Initialize \hat{x}⁽⁰⁾ suing

$${w}_{( n , k )}^{( 0 )}$$

in (2) and performing I₁
ADMM iterations.
Set t←1 (Initialize iteration counter) repeat
E-step: Update

$${w}_{( n , k )}^{( t )}$$

using \hat{x}^{(t-1)} in (1).
M-step: Estimate \hat{x}^{(t)} by using

$${w}_{( n , k )}^{( t )}$$

in (2) and
performing I₂ ADMM iterations.

t←t+1 (Increment iteration counter)

until

$${‖ {\hat{x}}^{( t )} - {\hat{x}}^{( t - 1 )} ‖}_{2}^{2} / {‖ {\hat{x}}^{( t - 1 )} ‖}_{2}^{2} < η ⋀ t > J return {\hat{x}}^{( t )}$$

### Image Comparison & Evaluation

Compressed sensing (CS) [38] is the predominant FRVCMR reconstruction method and widely used in both clinical and research settings, and was employed as the standard for comparison with EMORe. The CS image estimate, \hat{x}_{CS}, was obtained by solving a regularized weighted least-squares problem in (2) using binary participation weights derived from the initial SG-based bin assignments,

$${w}_{( n , k )}^{S G} = {w}_{( n , k )}^{( 0 )}$$

for all iterations, t.

$${\hat{x}}_{cs} = arg {min}_{x} {\sum }_{n = 1}^{N} {\sum }_{k = 1}^{K} [ \frac{{w}_{( n , k )}^{SG}}{L {σ}^{2}} {‖ {A}_{( n , k )} x - {y}_{n} ‖}_{2}^{2} ] + {λ}_{s} {‖ {∇}_{s} x ‖}_{1} + {λ}_{c} {‖ {∇}_{c} x ‖}_{1} + {λ}_{r} {‖ {∇}_{r} x ‖}_{1} \qquad ( 5 )$$

Similar to the EMORe, the ADMM algorithm was employed to solve the optimization problem in the CS reconstruction, with iterations terminating upon reaching a maximum count J or when the normalized squared difference between consecutive image iterates fell below a threshold η.

In phantom studies, where a reference image was available, we compared the quality of EMORe and CS images using the structural similarity index (SSIM ↑) [39] and peak signal-to-noise ratio (PSNR ↑), defined as 20 log₁₀

$$( \frac{max ( x )}{{‖ \hat{x} - x ‖}_{2} / \sqrt{N}} ) ( dB ) .$$

Additionally, the study measured blood-myocardium edge sharpness of all reconstructed images using edge sharpness assessment [40] presented for MRI. The edge sharpness (↑) was quantified as the mean slope of sigmoid functions fitted to pixel intensity profiles crossing the blood-myocardium boundaries, where residual motion artifacts typically introduce blurring. Higher slope values indicate improved edge sharpness and clearer delineation of anatomical structures. In the example 5D MRI data, a single blood-myocardium edge sharpness value was obtained by averaging estimates across all four respiratory states, measured on a fixed sagittal cardiac slice where the blood pool was fully enclosed by the myocardium. Furthermore, in studies where the true bin assignments were available, the study assessed the improvement in bin participation of data using EMORe by comparing initial and final Brier score [41], which evaluates the mean squared error of probabilistic bin assignments. The Brier score (↓) at EMORe iteration τ is defined as

$$\frac{1}{N} {\sum }_{n = 1}^{N} {\sum }_{k = 1}^{K} {( {\tilde{w}}_{( n , k )} - {w}_{( n , k )}^{( t )} )}^{2} ,$$

where \tilde{w}_{(n,k)} represents the true bin participation of readout n for bin k. In the absence of reference images for the in vivo study, two expert readers independently and blindly evaluated EMORe-CS cine slice pairs for perceived noise and artifacts quality using a 5-point Likert scale (5: excellent, 4: good, 3: fair, 2: poor, 1: non-diagnostic).

### Experimental Studies

To evaluate the robustness of the proposed EMORe reconstruction framework against uncompensated motion in 5D MRI, the study performed phantom and in vivo studies comparing EMORe to conventional CS reconstruction.

5D MRXCAT phantom study: The study simulated self-gated free-breathing 5D MRI scans using the MRXCAT phantom [42] from five digital subjects. Each subject comprised four respiratory phases (end-expiratory to end-inspiratory) and 20 cardiac phases spanning the full cardiac cycle, resulting in 4×20=80 cardiorespiratory phases. Respiratory periods ranged from 3.25 to 4.75 s across the subjects, with a random variation of 0 to 1 s introduced between consecutive cycles to mimic irregular breathing. Cardiac activity of the digital subjects was simulated with heart rates ranging between 62 and 83 bpm, with additional beat-to-beat variations of 0 to 160 ms in consecutive R-R intervals.

The k-space data were sampled using a pseudo-random Cartesian trajectory with self-gating [43], acquiring 75,000 readouts over a 5-minute scan with a repetition time (TR) of 4 ms. Coil sensitivity maps for an 8-channel array were generated using the Biot-Savart law, with coils positioned evenly in anterior and posterior planes. Complex circularly symmetric Gaussian noise was added to achieve a SNR of 30 dB. Additional imaging parameters are detailed in Table I.

TABLE 1
Imaging and Reconstruction Parameters
for Phantom and In Vivo 5D MRI Studies

| | | |
|---|---|---|
| Parameter | Phantom Study | In Vivo Study |

Imaging parameters

| | | |
|---|---|---|
| Number of Datasets | 50 | 13 |
| Spatial Resolution (mm) | 2 × 2 × 2 | 1.5-2.1 × 1.5-2.1 × |
| | | 2.0-2.8 |
| Matrix Size | 90 × 82 × 76 | 96 × 144 × 80 |
| Acceleration Rate R | 7.3 | 9.3-9.8 |
| Sex Distribution | 2/3 | 7/6 |
| Male/Female | | |
| Age Range (Mean), years | — | 20-68 (36) |
| BMI Range (Mean), kg/m² | — | 22-37 (28) |
| TE/TR (ms) | 1.2/4.0 | 1.2/3.0-3.2 |
| Sampling Pattern | 3D Cartesian | 3D Cartesian |
| Scan Time (min) | 5 | ~5 |

Reconstruction parameters

| | | |
|---|---|---|
| Regularization λₛ, λ_{c}, λᵣ | 1 × 10⁻³, 5 × 10⁻³, | 0.5 × 10⁻³, 2.5 × 10⁻³, |
| | 3 × 10⁻³ | 1.5 × 10⁻³ |
| Convergence threshold η | 10⁻⁴ | 10⁻⁴ |
| Outlier threshold τ | 3σ | 3σ |
| Prior α_{g}, αₒ | 0.85, 0.05 | 0.85, 0.05 |
| Inner iterations I₁, I₂ | 8, 4 | 8, 4 |
| Max outer iterations J | 240 | 240 |

To simulate sporadic bulk-motion artifacts, seven distinct outlier motion states were generated per subject. These included rigid-body translations of +20 mm along the superior-inferior (Z) axis and rotations of +10° about the anterior-posterior (Y) and superior-inferior (Z) axes, as well as a rotation of −10° about the left-right (X) axis. A positive rotation about the left-right (X) axis was omitted, reflecting the low likelihood of backward head-tilting motion in typical patient scenarios. For each of five digital subjects, ten experiments per subject were conducted to introduce varying levels of motion corruption: 0%, 5%, 10%, 15%, 20%, 30%, 40%, 50%, 60%, and 70%. Within each of these 50 simulated scans, the specified outlier corruption fraction was segmented into ten discrete motion instances, distributed throughout the acquisition using Poisson-disk sampling [44]. These motion events ranged in duration from approximately 1.5 s (in the 5% scenario) to 21 s (in the extreme 70% scenario). For each motion instance, one of the seven outlier motion states was randomly selected to replace the corresponding reference k-space data.

During data acquisition simulation, the SG signal was sampled every ten readouts in the superior-inferior direction. In the pre-preprocessing of the scanned data, the SG signal was reorganized into a Casorati matrix, and two band-pass filters with passbands of 0.1-0.5 Hz (respiratory) and 0.5-3 Hz (cardiac) were applied along the temporal dimension. Subsequently, principal component analysis (PCA) or independent component analysis (ICA) were performed on the filtered Casorati matrix to estimate respiratory and cardiac surrogate signals [13]. Based on these signals, k-space data were binned into four respiratory bins of equal data efficiency, each further divided into 20 cardiac bins by partitioning the R-R interval into equal-duration phases, resulting in K=80 cardiorespiratory bins.

The SG-based binary bin assignments were used to initialize the participation weights

$${w}_{( n , k )}^{( 0 )}$$

and prior θ_{(n,k)} using (3) and (4), respectively. Due to cardiac and respiratory variability, the binning is imperfect, even in the case of 0% outlier corruption fraction. An initial image estimate \hat{x}⁽⁰⁾ was obtained by partially solving (2) for I₁ ADMM iterations. Subsequently, the EMORe reconstruction iteratively performed the E-step and M-step to refine bin assignments, reject outlier data, and enhance image quality until convergence criteria were satisfied. For comparison, CS reconstructions were performed by solving (5) using fixed SG-based binary bin participation weights

$${w}_{( n , k )}^{S G} = {w}_{( n , k )}^{( 0 )} .$$

Image quality of EMORe and CS reconstructions was quantitatively assessed across all phantom experiments using PSNR, SSIM, and edge sharpness, using the true phantom images as reference. Additionally, improvement in bin assignments achieved by EMORe was evaluated against the initial SG-based binning employed in CS using the Brier score.

In vivo 5D MRI study: The study compared EMORe and CS reconstructions using in vivo 5D MRI data acquired from 13 healthy volunteers in a study approved by the institutional review board. Imaging was performed on a 3T clinical scanner (MAGNETOM Vida, Siemens Healthcare, Erlangen, Germany) equipped with a 48-channel receiver coil. Ferumoxytol-enhanced scans were obtained using a free-running, free-breathing acquisition with a fixed duration of approximately 5 minutes. Data were sampled using a pseudo-random Cartesian trajectory with SG. Detailed imaging parameters are summarized in Table I. To evaluate the robustness of EMORe in handling controlled bulk motion, three volunteers were instructed to simulate coughing during the final 30 seconds of their respective scans.

Acquired k-space data were retrospectively sorted into 80 cardiorespiratory bins using blind-source-separation, following methods described herein. For computational efficiency, coil compression via singular value decomposition was applied, reducing the original 30 acquisition coils to 8 virtual coils [45]. Coil sensitivity maps were estimated using the approach by Walsh et al. [46]. Subsequently, both EMORe and CS methods were applied to reconstruct 5D MRI images, using frameworks identical to those described for the phantom study described herein.

Due to the lack of a ground-truth reference for in vivo data, quantitative image assessment was limited to blood myocardium edge sharpness. For qualitative evaluation, blind scoring of artifacts was performed on EMORe and CS sagittal cine slice pairs from end-expiratory and end-inspiratory phases of each volunteer's reconstruction, yielding a total of 26 scored cine image pairs.

### Implementation Details

EMORe and CS reconstructions were implemented in MATLAB (Math Works, Natick, MA, USA) and executed on an NVIDIA H100 GPU at the Ohio Supercomputer Center. The average reconstruction times were approximately 23.7 minutes for EMORe and 19.1 minutes for CS in the phantom study, and 58.0 minutes for EMORe and 49.8 minutes for CS in the in vivo study.

### Results

### 5D MRI Phantom Study

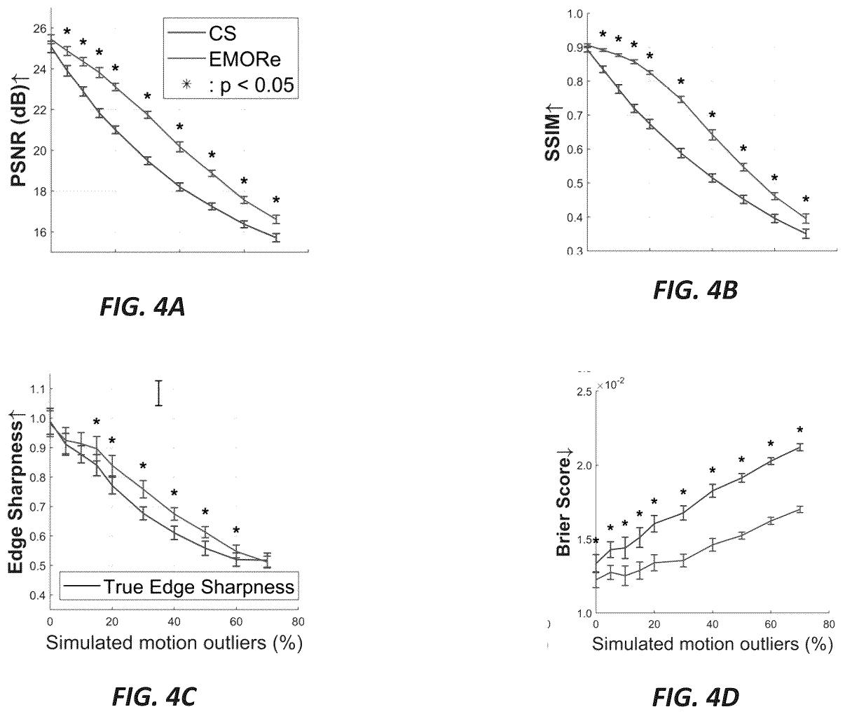

The quantitative comparisons between EMORe and CS reconstructions across five MRXCAT digital subjects at ten simulated outlier corruption levels are presented in FIGS. 4A-4D. Metrics reported include PSNR (FIG. 4A), SSIM (FIG. 4B), edge sharpness (FIG. 4C), and Brier score (FIG. 4D), averaged across subjects. Statistical significance was assessed using a paired ttest across subject at each outlier level, with significance indicated for p<0.05.

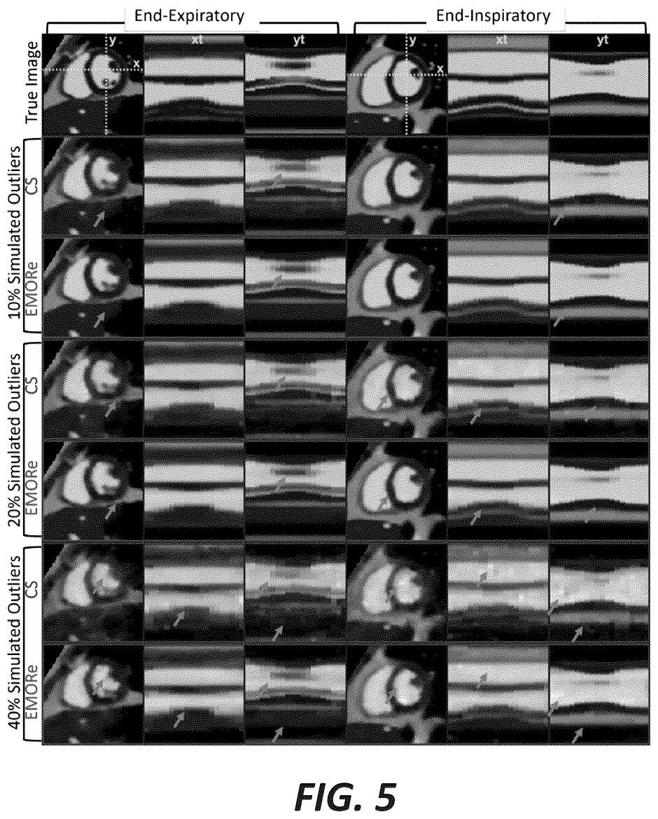

Representative end-expiratory and end-inspiratory short-axis cine slices from the ground truth, CS, and EMORe reconstructions are shown in FIG. 5 for three motion scenarios: 10%, 20%, and 40% simulated outliers. Corresponding temporal profiles along the x-t and y-t dimensions are also visualized to facilitate qualitative comparison of temporal consistency and boundary definition.

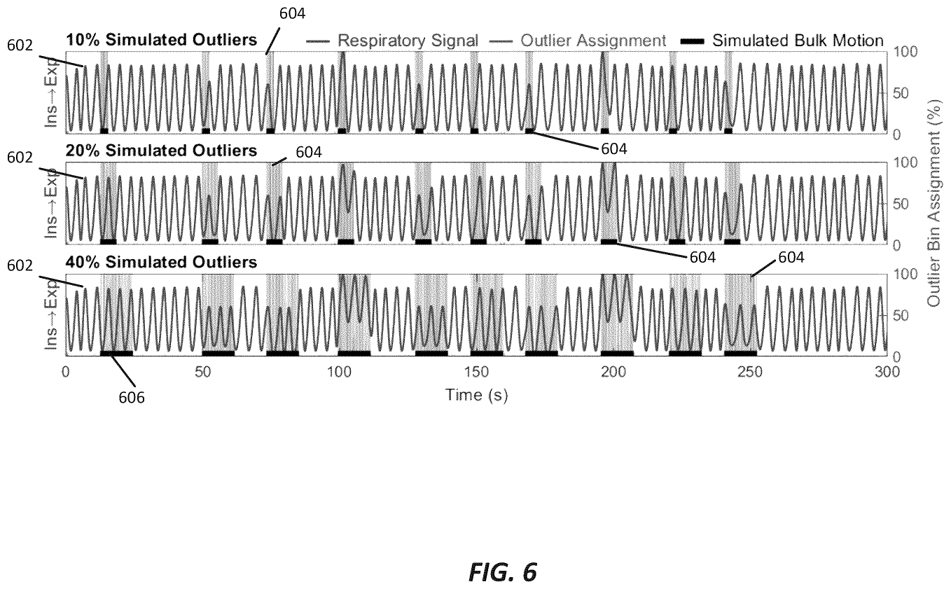

To evaluate EMORe's ability to reject corrupted data while preserving valid readouts, FIG. 6 includes respiratory signals 602, outlier assignments 604, and simulated bulk motion 606. Examples are shown for 10%, 20%, and 40% outlier scenarios, corresponding to the reconstructions in FIG. 5.

### 5D MRI in Vivo Study

The edge sharpness and the blind artifact scores comparisons between EMORe and CS for the 13 in vivo scans, including three scans with forced coughing in last 30 seconds, are summarized in Table II. EMORe demonstrated significantly improved (p<0.05) blood-myocardium edge sharpness compared to CS. Additionally, blind artifact scoring by two independent expert readers showed consistent and statistically significant improvements for EMORe relative to CS in perceived noise and artifact levels, using paired t-test (p<0.05) with Bonferroni correction.

TABLE II
Comparison of EMORE and CS for edge sharpness
and blind image scores for in-vivo study.

| | EMORe | CS |
|---|---|---|
| Metric | (Mean ± SEM) | (Mean ± SEM) |
| Edge sharpness (↑) | 0.722 ± 0.028* | 0.694 ± 0.023 |
| Blind artifact score | 4.00 ± 0.06* | 3.58 ± 0.13 |
| Reviewer 1 (↑) | | |
| Blind artifact score | 4.12 ± 0.14* | 3.65 ± 0.19 |
| Reviewer 2 (↑) | | |

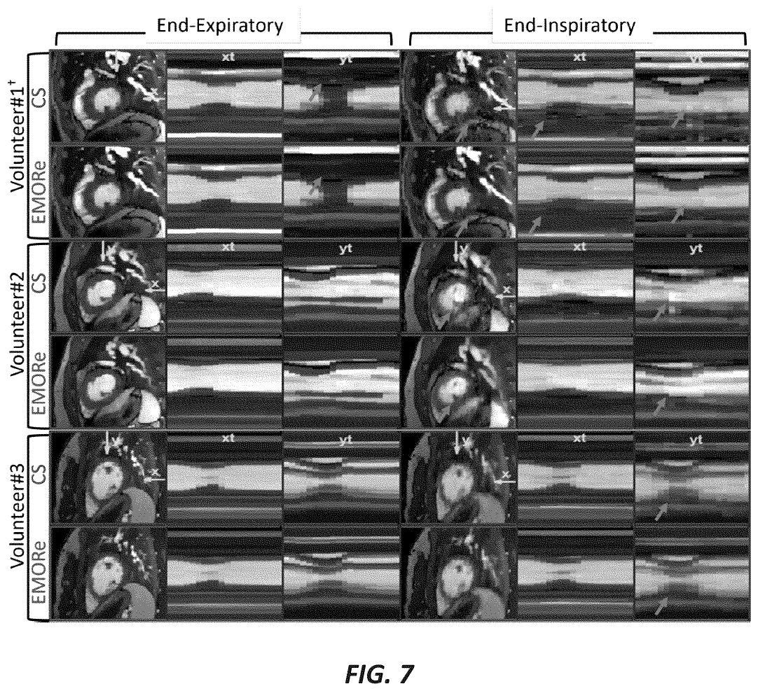

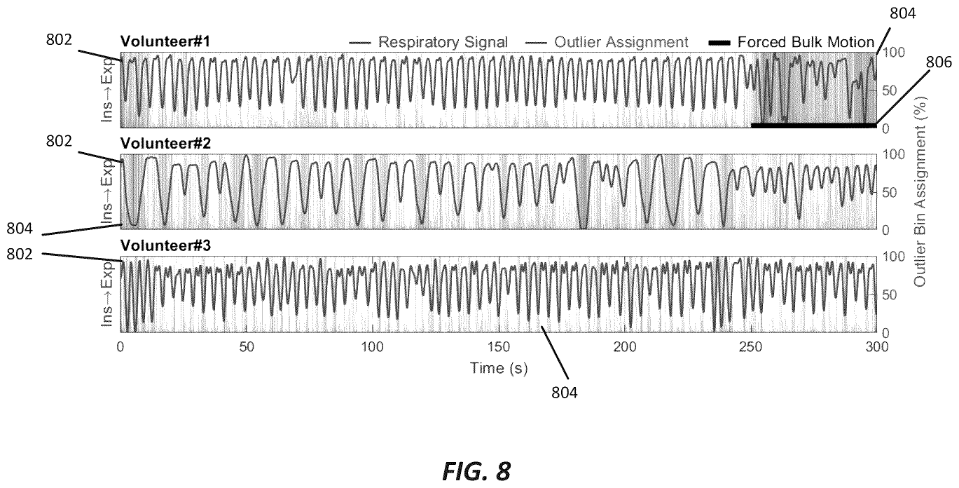

FIG. 7 presents sagittal cardiac frames and the corresponding horizontal and vertical temporal profiles at end-expiratory and end-inspiratory phases for EMORe and CS reconstructions from three representative volunteer scans. Volunteer #1 was instructed to simulate coughing motion during the final 30 seconds of the 5-minute scan, whereas Volunteers #2 and #3 underwent standard free-breathing acquisitions without specific instructions. FIG. 8 presents the respiratory surrogate signals 802 for the scans corresponding to FIG. 7; overlaid are forced bulk motion interval 806 and the outlier bin assignment percentage of the corresponding data 804.

### DISCUSSION

In the phantom experiments, EMORe image quality was indistinguishable from CS in the absence of motion outliers, as measured by PSNR, SSIM, and edge sharpness. Yet even without bulk motion outliers, EMORe achieved statistically significant improvement in the Brier score by adaptively refining a soft binning; this highlights that some inaccuracies in self-gating-based binning may occur without bulk motion, due to irregular breathing and beat-to-beat variations. With increasing simulated bulk motion outliers, CS reconstructions exhibited significant degradation, whereas EMORe maintained higher fidelity, reflecting its robustness against sporadic motion artifacts. In extreme cases with more than 40% motion outliers, although EMORe maintained an advantage over CS, the overall image quality of EMORe also degrades substantially, highlighting the limitations of the proposed method under widespread data corruption.

Visual comparisons in FIG. 5 further support these quantitative findings in the phantom study, showing that EMORe reconstructions exhibit clearer anatomical boundaries and reduced motion artifacts relative to CS, particularly at higher outlier levels. Moreover, the respiratory surrogate plots in FIG. 6 demonstrate EMORe's capability to aggressively reject outlier data during simulated bulk motion instances, resulting in the improved Brier scores and preserving the fidelity of data in the valid motion state bins throughout the scan.

From the in-vivo study, EMORe reconstructions showed statistically significant improvement in blood-myocardium edge sharpness, compared to CS recovery without adaptive refinement of bin assignments. Likewise, the expert reviewers' 1-5 Likert scores, averaged across cine image pairs (N=26), improved by 0.45 via EMORe processing, showing enhanced robustness to motion artifacts. Together, these improvements suggest practical value in clinical application.

Among the 13 volunteers, 3 were instructed to perform intermittent coughing to provide a controlled evaluation of EMORe's ability to identify and discard data readouts corrupted by sporadic bulk motion. Results in FIG. 8 showed aggressive rejection of corrupted readouts during the known coughing interval.

The adaptive binning in the EMORe framework is achieved at a modest computational overhead, as reflected in the reconstruction time increases of approximately 24% and 16% for phantom and in vivo datasets, respectively. Moreover, EMORe required the selection of additional hyperparameters, including the prior weights α_{g} and αₒ, as well as the outlier rejection threshold t, to balance the trade-offs among artifact suppression, outlier rejection, and algorithm stability.

Future directions include deep-learning for image estimation in the M-step to reduce computation time Additionally, extending EMORe to in vivo 5D flow studies could enhance quantitative flow analysis and broaden its clinical applicability. Furthermore, evaluating EMORe in patient populations with irregular breathing, arrhythmias, or fidgeting movement would provide further validation of its robustness in clinical settings.

Volumetric cardiovascular magnetic resonance imaging (CMR) allows a comprehensive assessment of the whole heart, capturing the 3D volume in different motion phases. Volumetric imaging circumvents the major limitations of standard 2D CMR, such as breath hold requirement, inability to capture out-of-plane motion, limited slice thickness, and the need for extensive preplanning [1]. Standard volumetric CMR is performed for several minutes under electrocardiogram (ECG) guidance and prospective respiratory gating using navigator echoes to resolve cardiac and respiratory motion in the scan data [2]. However, depending on the breathing pattern and the extent of arrhythmia, this approach may lead to unpredictably long acquisition times, sometimes exceeding ten minutes [3]. In addition, navigator echoes disrupt the steady-state of magnetization and thus are not compatible with several common CMR pulse sequences [4]. As an alternative to navigator echoes, respiratory bellows [5] can be used for prospective or retrospective gating, but their use has been limited due to inconsistent performance [6]. Moreover, ECG triggering is unreliable at mid- to high-field strengths due to magnetohydrodynamic effects and magnetic-field-induced distortions [7]. Therefore, the widespread clinical adoption of volumetric CMR has been limited due to long scan times [8] and the challenge of compensating for the multi dynamic motion of the heart [4]. More recently, there has been increased interest in free-running self-gated volumetric imaging (FRVCMR) [9]-[13], in which data are collected continuously for several minutes without the guidance of navigator echoes, respiratory bellows, or even ECG. Furthermore, FRV-CMR offers flexibility in retrospectively controlling temporal resolution by selecting the number of cardiac and respiratory bins after the scan [14].

In self-gated acquisition [15], a central phase encoding readout is periodically acquired to augment k-space data acquisition. Changes in self-gating readouts are attributed to physiological motion. A common approach for extracting respiratory and cardiac signals relies on blind source separation. These surrogate motion signals are employed to assign the data into motion bins, leading to motion resolved volumetric CMR [16]. There are various approaches to retrospectively binning the cardio-respiratory data, according to clinical application and computational constraints. The first approach is respiratory-compensated (by keeping data from only one respiratory phase) and cardiac-resolved binning [17]-[19]. The limitation of this approach is that data efficiency is 50% or lower because the data from only one respiratory phase are utilized. The second approach is respiratory-corrected (by registering or deforming all other respiratory phases to one reference phase) and cardiac-resolved binning [20]-[22]. Although this approach provides 100% data efficiency, it requires estimation of high-quality deformation fields, which is a difficult task; moreover, this approach averages the effect of respiratory phases on cardiac function. The third and more recent approach is cardio-respiratory-resolved binning, commonly known as 5D MRI [16], [23], [24]. There has been increased interest in 5D MRI among researchers and clinicians due to its 100% imaging efficiency, ability to resolve respiratory effects on cardiac function [25], and introduction of a respiratory dimension for enhanced temporal regularization [11], [26]. Nevertheless, the use of self-gating and blind-source-separation in all three approaches assumes periodic cardiac and respiratory motion signals; yet, perfectly periodic motion is not realized in clinical settings due to patient movement, presence of beat-to-beat variability, or inconsistent breathing patterns [27]. Therefore, in all three binning approaches, motion suppression is degraded by inaccuracies introduced both by the signal extraction process and by motion outliers due to involuntary bulk motion such as deep breaths, coughing, sneezing, or twitches. These inaccuracies lead to incorrect assignment of k-space data to motion bins, resulting in residual motion artifacts and image blurring. Although Pilot Tone (PT) provides an alternative to self-gating, the extraction of physiological motion from PT faces similar challenges.

Conventional methods on FRV-CMR reconstruction based on compressive recovery [13], [26], or deep learning models do not account for the discussed imperfections in motion estimation, thereby limiting the quality of recovered images. Residual motion from imperfect retrospective binning can arise from two categories: valid data assigned to a wrong motion bin by inaccurate motion estimation; and, motion outlier data corrupted by sporadic bulk motion. For outlier rejection, static imaging protocols commonly employ motion tracking with fixed thresholds or similarity metrics [31]-[33]. However, these approaches lack adaptability to the dynamic spatiotemporal correlations inherent in CMR. Recent advances in outlier rejection in dynamic imaging leverage physics-guided group sparsity or use of expectation-maximization (EM) to determine inliers and outliers using a mixture model of the two classes [35]. However, these outlier rejection techniques suffer from the major limitation of discarding incorrectly binned, yet valid, data as outliers and effectively increasing the acceleration rate, as they lack the flexibility to correct inaccurate bin assignments. There is recent work on intra-bin motion correction proposing rigid motion correction within a respiratory bin to reduce respiratory blurring in 5D MRI. While that method improves sharpness within respiratory bins, it also assumes perfect signal extraction from self-gating and accurate initial bin assignments.

The example implementation includes expectation maximization (EM)-guided binning correction with outlier rejection (EMORe), integrates adaptive outlier rejection and binning refinement within a single iterative reconstruction framework using EM. Unlike previous works, EMORe does not assume self-gating-based binning as final, but rather leverages intra-bin inconsistencies to iteratively reassign data readouts to their correct motion bins or to reject a readout to an outlier bin, with corresponding improvement in image estimates. In sum, the study described herein validated the advantages of the EMORe reconstruction algorithm over conventional reconstruction in a 5D MRXCAT phantom study, and showed the improvement of EMORe in an in-vivo 5D MRI reconstruction compared to a conventional technique.

### REFERENCES

[1] P. Dyverfeldt, M. Bissell, A. J. Barker, A. F. Bolger, C.-J. Carlhäll, T. Ebbers, C. J. Francios, A. Frydrychowicz, J. Geiger, D. Giese, et al., “4D flow cardiovascular magnetic resonance consensus statement,” J. Cardiovasc. Magn. Reson., vol. 17, no. 1, p. 72, 2015.
[2] M. Salerno, B. Sharif, H. Arheden, A. Kumar, L. Axel, D. Li, and S. Neubauer, “Recent advances in cardiovascular magnetic resonance: techniques and applications,” Circulation: Cardiovascular Imaging, vol. 10, no. 6, p. e003951, 2017.
[3] H. W. Goo, “Comparison between three-dimensional navigator gated whole-heart MRI and two-dimensional cine MRI in quantifying ventricular volumes,” Korean J. Radiol., vol. 19, no. 4, pp. 704-714, 2018.
[4] M. Zaitsev, J. Maclaren, and M. Herbst, “Motion artifacts in MRI: A complex problem with many partial solutions,” J. Magn. Reson. Imaging, vol. 42, no. 4, pp. 887-901, 2015.

[5] Q. Yuan, L. Axel, E. H. Hernandez, L. Dougherty, J. J. Pilla, C. H. Scott, V. A. Ferrari, and A. S. Blom, “Cardiac-respiratory gating method for magnetic resonance imaging of the heart,” Magn. Reson. Med., vol. 43, no. 2, pp. 314-318, 2000.
[6] C. Santelli, R. Nezafat, B. Goddu, W. J. Manning, J. Smink, S. Kozerke, and D. C. Peters, “Respiratory bellows revisited for motion compensation: preliminary experience for cardiovascular MR,” Magn. Reson. Med., vol. 65, no. 4, pp. 1097-1102, 2011.
[7] M. Jekic, Y. Ding, R. Dzwonczyk, P. Burns, S. V. Raman, and O. P. Simonetti, “Magnetic field threshold for accurate electrocardiogram the MRI environment,” Magn. Reson. Med., vol. 64, no. 6, pp. 1586-1591, 2010.
[8] M. M. Bissell, F. Raimondi, L. Ait Ali, B. D. Allen, A. J. Barker, A. Bolger, N. Burris, C.-J. Carhäll, J. D. Collins, T. Ebbers, et al., “4D flow cardiovascular magnetic resonance consensus statement: 2023 update,” J. Cardiovasc. Magn. Reson., vol. 25, no. 1, p. 40, 2023.
[9] L. Feng, L. Axel, H. Chandarana, K. T. Block, D. K. Sodickson, and R. Otazo, “XD-GRASP: golden-angle radial MRI with reconstruction of extra motion-state dimensions using compressed sensing,” Magn. Reson. Med., vol. 75, no. 2, pp. 775-788, 2016.
[10] L. Di Sopra, D. Piccini, S. Coppo, M. Stuber, and J. Yerly, “An automated approach to fully self-gated free-running cardiac and respiratory motion-resolved 5D whole-heart MRI,” Magn. Reson. Med., vol. 82, no. 6, pp. 2118-2132, 2019.
[11] L. E. Ma, J. Yerly, D. Piccini, L. Di Sopra, C. W. Roy, J. C. Carr, C. K. Rigsby, D. Kim, M. Stuber, and M. Markl, “5D flow MRI: a fully self-gated, free-running framework for cardiac and respiratory motion-resolved 3D hemodynamics,” Radiology: Cardiothoracic Imaging, vol. 2, no. 6, p. e200219, 2020.
[12] T. Küstner, A. Bustin, O. Jaubert, R. Hajhosseiny, P. G. Masci, R. Neji, R. Botnar, and C. Prieto, “Fully self-gated free-running 3D cartesian cardiac cine with isotropic whole-heart coverage in less than 2 min,” NMR in Biomedicine, vol. 34, no. 1, p. e4409, 2021.
[13] A. Pruitt, A. Rich, Y. Liu, N. Jin, L. Potter, M. Tong, S. Rajpal, O. Simonetti, and R. Ahmad, “Fully self-gated whole-heart 4D flow imaging from a 5-minute scan,” Magn. Reson. Med., vol. 85, no. 3, pp. 1222-1236, 2021.
[14] R. J. Holtackers and M. Stuber, “Free-running cardiac and respiratory motion-resolved imaging: A paradigm shift for managing motion in cardiac MRI?,” Diagnostics, vol. 14, no. 17, p. 1946, 2024.
[15] A. C. Larson, R. D. White, G. Laub, E. R. Mc Veigh, D. Li, and O. P. Simonetti, “Self-gated cardiac cine MRI,” Magn. Reson. Med., vol. 51, no. 1, pp. 93-102, 2004.
[16] L. Feng, S. Coppo, D. Piccini, J. Yerly, R. P. Lim, P. G. Masci, M. Stuber, D. K. Sodickson, and R. Otazo, “5D whole-heart sparse MRI,” Magn. Reson. Med., vol. 79, no. 2, pp. 826-838, 2018.
[17] J. Liu, P. Spincemaille, N. C. Codella, T. D. Nguyen, M. R. Prince, and Y. Wang, “Respiratory and cardiac self-gated freebreathing cardiac cine imaging with multi-echo 3D hybrid radial SSFP acquisition,” Magn. Reson. Med., vol. 63, no. 5, pp. 1230-1237, 2010.
[18] M. Usman, B. Ruijsink, M. Nazir, G. Cruz, and C. Prieto, “Free breathing whole-heart 3D cine MRI with self-gated Cartesian trajectory,” Magn. Reson. Imaging, vol. 38, pp. 129-137, 2017.
[19] M. H. Moghari, A. Barthur, M. E. Amaral, T. Geva, and A. J. Powell, “Free-breathing whole-heart 3D cine magnetic resonance imaging with prospective respiratory motion compensation,” Magn. Reson. Med., vol. 80, no. 1, pp. 181-189, 2018.
[20] C. Stehning, P. Börnert, K. Nehrke, H. Eggers, and M. Stuber, “Free-breathing whole-heart coronary MRA with 3D radial SSFP and self-navigated image reconstruction,” Magn. Reson. Med., vol. 54, no. 2, pp. 476-480, 2005.
[21] H. Bhat, L. Ge, S. Nielles-Vallespin, S. Zuehlsdorff, and D. Li, “3D radial sampling and 3D affine transform-based respiratory motion correction technique for free-breathing whole-heart coronary MRA with 100% imaging efficiency,” Magn. Reson. Med., vol. 65, no. 5, pp. 1269-1277, 2011.
[22] R. R. Ingle, H. H. Wu, N. O. Addy, J. Y. Cheng, P. C. Yang, B. S. Hu, and D. G. Nishimura, “Nonrigid autofocus motion correction for coronary MR angiography with a 3D cones trajectory,” Magn. Reson. Med., vol. 72, no. 2, pp. 347-361, 2014.
[23] H. H. Wu, D. G. Nishimura, B. S. Hu, and M. V. McConnell, “Acquisition and visualization of 5D respiratory-resolved cardiac MRI,” J. Cardiovasc. Magn. Reson., vol. 14, no. Suppl 1, p. P237, 2012.
[24] X. Sieber, P. S. Chandrasekaran, J. Varghese, Y. Liu, C. Roy, J. Yerly, S. M. Arshad, R. Ahmad, R. B. Van Heeswijk, M. Stuber, et al., “Ferumoxytol-enhanced free-running 5D whole-heart CMR at 0.55 T,” J. Cardiovasc. Magn. Reson., vol. 27, 2025.
[25] E. K. Weiss, J. Baraboo, C. K. Rigsby, J. D. Robinson, L. Ma, M. B. Falcão, C. W. Roy, M. Stuber, and M. Markl, “Respiratory-resolved five-dimensional flow cardiovascular magnetic resonance: In-vivo validation and respiratory-dependent flow changes in healthy volunteers and patients with congenital heart disease,” J. Cardiovasc. Magn. Reson., vol. 26, no. 2, p. 101077, 2024.
[26] L. Feng, L. Axel, H. Chandarana, K. T. Block, D. K. Sodickson, and R. Otazo, “XD-GRASP: golden-angle radial MRI with reconstruction of extra motion-state dimensions using compressed sensing,” Magn. Reson. Med., vol. 75, no. 2, pp. 775-788, 2016.
[27] S. M. Arshad, L. C. Potter, C. Chen, Y. Liu, P. Chandrasekaran, C. Crabtree, M. S. Tong, O. P. Simonetti, Y. Han, and R. Ahmad, “Motion-robust free-running volumetric cardiovascular MRI,” Magn. Reson. Med., vol. 92, no. 3, pp. 1248-1262, 2024.
[28] J. Ludwig, P. Speier, F. Seifert, T. Schaeffter, and C. Kolbitsch, “Pilot tone-based motion correction for prospective respiratory compensated cardiac cine MRI,” Magn. Reson. Med., vol. 85, no. 5, pp. 2403-2416, 2021.
[29] C. W. Roy, B. Milani, J. Yerly, S. Si-Mohamed, L. Romanin, A. Bustin, E. Tenisch, T. Rutz, M. Prsa, and M. Stuber, “Intrabin correction and inter-bin compensation of respiratory motion in free-running five-dimensional whole-heart magnetic resonance imaging,” J. Cardiovasc. Magn. Reson., vol. 26, no. 1, p. 101037, 2024.
[30] T. Küstner, N. Fuin, K. Hammernik, A. Bustin, H. Qi, R. Hajhosseiny, P. G. Masci, R. Neji, D. Rueckert, R. M. Botnar, et al., “CINENet: deep learning-based 3D cardiac cine MRI reconstruction with multi-coil complex-valued 4D spatio-temporal convolutions,” Sci. Rep., vol. 10, no. 1, p. 13710, 2020.
[31] M. Niethammer, S. Bouix, S. Aja-Fernández, C.-F. Westin, and M. E. Shenton, “Outlier rejection for diffusion weighted imaging,” in International Conference on Medical Image Computing and Computer-Assisted Intervention, pp. 161-168, Springer, 2007.
[32] S. Dolui, Z. Wang, R. T. Shinohara, D. A. Wolk, J. A. Detre, and A. D. N. Initiative, “Structural correlation-based outlier rejection (SCORE) algorithm for arterial spin labeling time series,” J. Magn. Reson. Imaging, vol. 45, no. 6, pp. 1786-1797, 2017.
[33] A. X. Patel, P. Kundu, M. Rubinov, P. S. Jones, P. E. Vértes, K. D. Ersche, J. Suckling, and E. T. Bullmore, “A wavelet method for modeling and despiking motion artifacts from resting-state fMRI time series,” Neuroimage, vol. 95, pp. 287-304, 2014.
[34] G. J. McLachlan and T. Krishnan, The EM Algorithm and Extensions. John Wiley & Sons, 2008.
[35] J. F. van Amerom, D. F. Lloyd, A. N. Price, M. Kuklisova Murgasova, P. Aljabar, S. J. Malik, M. Lohezic, M. A. Rutherford, K. Pushparajah, R. Razavi, et al., “Fetal cardiac cine imaging using highly accelerated dynamic MRI with retrospective motion correction and outlier rejection,” Magn. Reson. Med., vol. 79, no. 1, pp. 327-338, 2018.
[36] S. Boyd, N. Parikh, E. Chu, B. Peleato, J. Eckstein, et al., “Distributed optimization and statistical learning via the alternating direction method of multipliers,” Foundations and Trends® in Machine Learning, vol. 3, no. 1, pp. 1-122, 2011.
[37] S. N. Kadir, D. F. Goodman, and K. D. Harris, “Highdimensional cluster analysis with the masked EM algorithm,” Neural Computation, vol. 26, no. 11, pp. 2379-2394, 2014.
[38] M. Lustig, D. L. Donoho, J. M. Santos, and J. M. Pauly, “Compressed sensing MRI,” IEEE Signal Process. Mag., vol. 25, no. 2, pp. 72-82, 2008.
[39] Z. Wang, A. C. Bovik, H. R. Sheikh, and E. P. Simoncelli, “Image quality assessment: from error visibility to structural similarity,” IEEE Trans. Image Process., vol. 13, no. 4, pp. 600-612, 2004.
[40] R. Ahmad, Y. Ding, and O. P. Simonetti, “Edge sharpness assessment by parametric modeling: application to magnetic resonance imaging,” Concepts Magn. Reson., Part A, vol. 44, no. 3, pp. 138-149, 2015.
[41] G. W. Brier, “Verification of forecasts expressed in terms of probability,” Monthly Weather Review, vol. 78, no. 1, pp. 1-3, 1950.
[42] L. Wissmann, C. Santelli, W. P. Segars, and S. Kozerke, “MRXCAT: Realistic numerical phantoms for cardiovascular magnetic resonance,” J. Cardiovasc. Magn. Reson., vol. 16, pp. 1-11, 2014.
[43] M. Joshi, A. Pruitt, C. Chen, Y. Liu, and R. Ahmad, “Technical report (v1. 0)—pseudo-random Cartesian sampling for dynamic MRI,” arXiv preprint arXiv: 2206.03630, 2022.
[44] R. L. Cook, “Stochastic sampling in computer graphics,” ACM Trans. Graph., vol. 5, no. 1, pp. 51-72, 1986.
[45] M. Buehrer, K. P. Pruessmann, P. Boesiger, and S. Kozerke, “Array compression for MRI with large coil arrays,” Magn. Reson. Med., vol. 57, no. 6, pp. 1131-1139, 2007.
[46] D. O. Walsh, A. F. Gmitro, and M. W. Marcellin, “Adaptive reconstruction of phased array MR imagery,” Magn. Reson. Med., vol. 43, pp. 682-690, May 2000.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A computer-implemented method for image reconstruction, the method comprising:
receiving k-space data from a magnetic resonance imaging (MRI) machine, the k-space data comprising a plurality of k-space readouts;
sorting the k-space readouts into a plurality of bins comprising binned k-space data, each of the plurality of bins corresponding to a respective phase of a cardiac and respiratory cycle;
iteratively performing the steps of:
(i) computing a soft participation weight for each k-space readout, wherein the soft participation weight reflects a posterior probability that each k-space readout belongs to each of the plurality of bins; and
(ii) updating an image estimate by solving a weighted optimization problem;
determining when a convergence criterion is reached; and outputting a motion-resolved volumetric MRI image when the convergence criterion is reached.

2. The computer-implemented method of claim 1, wherein step (ii) comprises fixed ADMM iterations.

3. The computer-implemented method of claim 1, wherein the convergence criterion comprises a maximum number of ADMM iterations.

4. The computer-implemented method of claim 1, wherein the convergence criterion comprises a threshold of normalized squared image difference between iterations of steps (i) and (ii).

5. The computer-implemented method of claim 1, wherein the k-space data comprises motion artifacts originating from respiratory, cardiac, or bulk motion.

6. The computer-implemented method of claim 1, wherein the plurality of k-space readouts are acquired by self-gating readouts.

7. A system comprising:
an MRI machine;
a controller operably coupled to the MRI machine, wherein the controller comprises a processor and a memory, wherein the memory has non-transitory computer-readable instructions stored thereon, that, when executed by the processor, cause the processor to:
receive k-space data from a magnetic resonance imaging (MRI) machine, the k-space data comprising a plurality of k-space readouts;
sort the k-space readouts into a plurality of bins comprising binned k-space data, each of the plurality of bins corresponding to a respective phase of a respiratory cycle; iteratively performing the steps of:
(i) computing a soft participation weight for each k-space readout, wherein the soft participation weight reflects a posterior probability that each k-space readout belongs to each of the plurality of bins; and
(ii) updating an image estimate by solving a weighted optimization problem; determining a convergence criterion is reached; and
output a motion-resolved volumetric MRI image when the convergence criterion is reached.

8. The system of claim 7, wherein step (ii) comprises fixed ADMM iterations.

9. The system of claim 7, wherein the convergence criterion comprises a maximum number of ADMM iterations.

10. The system of claim 7, wherein the convergence criterion comprises a threshold of normalized squared image difference between iterations of steps (i) and (ii).

11. The system of claim 7, wherein the k-space data comprises motion artifacts originating from respiratory, cardiac, or bulk motion.

12. The system of claim 7, wherein the plurality of k-space readouts are acquired by self-gating readouts.

13. The system of claim 7, wherein the system further comprises a graphical user interface configured to display the motion-resolved volumetric MRI image.

14. The system of claim 7, wherein the system further comprises a remote computing device, and wherein the instructions further cause the processor to transmit the motion-resolved volumetric MRI image to the remote computing device.

15. A non-transitory computer-readable medium having instructions thereon, that, when executed by a processor, cause the processor to:
receive k-space data from a magnetic resonance imaging (MRI) machine, the k-space data comprising a plurality of k-space readouts;
sort the k-space readouts into a plurality of bins comprising binned k-space data, each of the plurality of bins corresponding to a respective phase of a respiratory cycle;
iteratively performing the steps of:
(i) computing a soft participation weight for each k-space readout, wherein the soft participation weight reflects a posterior probability that each k-space readout belongs to each of the plurality of bins; and
(ii) updating an image estimate by solving a weighted optimization problem;
determine a convergence criterion is reached; and
output a motion-resolved volumetric MRI image when a convergence criterion is reached.

16. The non-transitory computer-readable medium of claim 15, wherein step (ii) comprises I2 ADMM iterations.

17. The non-transitory computer-readable medium of claim 15, wherein the convergence criterion comprises a maximum number of ADMM iterations.

18. The non-transitory computer-readable medium of claim 15, wherein the convergence criterion comprises a threshold of normalized squared image difference between iterations of steps (i) and (ii).

19. The non-transitory computer-readable medium of claim 15, wherein the k-space data comprises motion artifacts originating from respiratory, cardiac, or bulk motion.

20. The non-transitory computer-readable medium of claim 15, wherein the plurality of k-space readouts are acquired by self-gating readouts.
